(19) United States
(12) Patent Application Publication
(10) Pub. No.: US 2017/0105654 A1
(43) Pub. Date: Apr. 20, 2017

(54) DEVICE FOR MONITORING THE FUNCTION OF SPHINCTERS IN THE GASTROINTESTINAL TRACT AND METHOD OF USE

(71) Applicant: UTI Limited Partnership, Calgary (CA)

(72) Inventors: Dan Sadowski, Edmonton (CA); Orly Yadid-Pecht, Calgary (CA); Qian Lu, Calgary (CA); Martin P. Mintchev, Calgary (CA)

(73) Assignee: UTI Limited Partnership, Calgary, AB (CA)

(21) Appl. No.: 15/317,951

(22) PCT Filed: Jun. 15, 2015
(86) PCT No.: PCT/IB15/54527

§ 371 Date: Dec. 11, 2016

Related U.S. Application Data
(60) Provisional application No. 62/011,907, filed on Jun. 13, 2014.

(51) Int. Cl. A61B 5/07; A61B 5/00; A61B 8/12

(52) U.S. Cl. CPC A61B 5/07; A61B 8/12; A61B 5/4233; A61B 5/0084; A61B 5/0086; A61B 5/742

(57) ABSTRACT

The present invention provides a device and a method for monitoring the function of sphincters in the gastrointestinal (GI) tract. The device of the invention includes a signal-generating device that is placed at one side of a GI sphincter, and a signal-detecting device that is placed at the other side of the GI sphincter whose function is to be monitored. The device detects and analyzes the change in the characteristics of the signals that are generated by the signal-generating device and detected by the signal-detecting device. In this manner, the signal data detected during the opening and closing of the GI sphincter can be stored in a data logger and/or can be monitored in real time.

### CROSS-REFERENCE TO RELATED APPLICATIONS

This application claims the priority benefit of U.S. Provisional Application No. 62/011,907, filed Jun. 13, 2014, which is incorporated herein by reference in its entirety. Because Jun. 13, 2015, is a Saturday, the due date for filing this PCT Application is the next business day, Monday, Jun. 15, 2015.

### FIELD OF THE INVENTION

The present invention relates to a device and a method for monitoring the function of sphincters in the gastrointestinal (GI) tract. The device of the invention includes a signal-generating device that is placed at one side of a GI sphincter, and a signal-detecting device that is placed at the other side of the GI sphincter whose function is to be monitored. The device detects and analyzes the change in the characteristics of the signals that is generated by the signal-generating device and detected by the signal-detecting device. The wave data detected during the opening and closing of the GI sphincter can be stored in a data logger and/or can be monitored in real time.

### BACKGROUND OF THE INVENTION

Human gastrointestinal (GI) tract spans from the mouth to the anus, and includes the pharynx, the esophagus, the stomach, the small and large intestines and the rectum. Along the way, ring-like muscle fibers called sphincters control the passage of content from one specialized portion of the GI tract to another. For example, the lower esophageal sphincter (LES) controls the opening between the esophagus and the stomach. The malfunction of GI sphincters result in a variety of disorders and diseases. For example, the malfunction of the upper esophageal sphincter (UES) can cause aspiration of gastric contents into the lungs and the airways as well as regurgitation of stomach contents into the pharynx and the larynx, leading to complaints and disorders such as pneumonia, bronchitis and laryngitis, usually associated with the entry of gastric contents above and beyond the esophagus. Moreover, a significant percent of deaths has been attributed to aspiration of gastric contents.

Gastroesophageal reflux disease (GERD) is considered the most common GI disease, affecting an estimated two percent of the adult population. Without being bound by any theory, it is believed that the main cause of GERD is a malfunction of the LES, which no longer prevents the contents of the stomach from refluxing into the esophagus, resulting in various esophageal symptoms and mucosal damage. It has been hypothesized that such esophageal damage may in some instances be a precursor to esophageal cancer.

The pyloric sphincter, another ring-like muscle, eventually relaxes to allow stomach contents to enter the first part of the small intestine, the duodenum. If the pyloric sphincter does not contract and relax properly, the stomach contents may be pushed down prematurely into the intestine causing indigestion, or they can be retained longer in the stomach, causing delayed gastric emptying.

The ileocecal valve (ICV) is a sphincter-type valve between the small intestine and the large intestine, the function of ICV is to prevent the backwards flow of waste from the large intestine into the small intestine. An open ICV are extremely common in today's society and its symptoms are often misdiagnosed.

Dysfunction of the anal sphincter leads to fecal incontinence, i.e., loss of voluntary control of the sphincter to retain stool in the rectum. Fecal incontinence is frequently a result of childbearing injuries or prior anorectal surgery.

Several methods have been developed to monitor the function of different GI sphincters, including issued patents, patent application and scientific papers. One example is a manometry apparatus for measuring the esophageal sphincter as described in U.S. Pat. No. 6,773,452. In one preferred embodiment of this method includes a catheter having a balloon that is located and inflated within the esophagus to trigger esophageal motility. A second, non-elastic balloon is positioned within the LES, and a third balloon is inflated within the stomach to help align the second balloon with the LES. By repeating the inflations of the second balloon with increasing volumes of air to obtain an increased physiological response, a volume vs. pressure curve can be established for a particular patient that allows the assessment of LES function. The invention can also be used to measure the compliance of the anal sphincter.

U.S. Pat. No. 5,117,827 describes a catheter including a flexible tube having a pH probe disposed distally and a pressure sensor disposed a known distance proximally to monitor gastric acid reflux. In one embodiment, the probe is placed within the stomach of the patient, and it is withdrawn until the LES respiration phasic pressure is observed on the pressure display. Next, the probe is withdrawn at known distance. A relationship between different positions of the probe and their corresponding pressures can be obtained, which can be utilized to monitor the function of the LES as well as gastric acid reflux in a patient.

In U.S. patent application publication number 2008/0033274, a preferred embodiment is reported to monitor naturally evolved hydrogen gas in the human stomach to detect the transient lower esophageal sphincter relaxations (TLESRs). Concentration of the gas is recorded during the time of the measurement by a sensor positioned about 5 cm above the LES. The functioning of the LES is characterized inter alia by the frequency of the occurrences of transient lower esophageal sphincter relaxations (number of TLESRs in a defined time period), slope of the onset and of the offset, levels and distances between two concentration peaks of the marker gas detected by the sensor versus time.

Another mechanical measurement of sphincters and narrowing regions in hollow biological organs is disclosed in U.S. patent application publication no. 2008/0161730. The apparatus comprises a catheter with an inflatable balloon to be inserted and inflated in a narrow region, including sphincters. In a preferred embodiment, multiple sets of electrodes are provided inside the balloon and cross-sectional recordings are made based on measurements of the electrical impedance of an electrically conducting fluid in a three-dimensional profile inside the balloon.

Indirect methods of assessing GI sphincters are also available. A method for diagnosing motility disorders of a gastrointestinal tract is proposed in U.S. patent application publication no. 2013/0046150. The method includes recording electrical signal from the GI tract by a portable electronic device carried by the patient in real time in assessing the bloating condition, which can be caused by gastroesophageal reflux disease. In one embodiment, an array of external electrodes on a support structure for recording signals from the esophagus, stomach and duodenum is placed on the human body for real time data collection.

Another method for diagnosing relaxation of the LES is based on a micro-manometric technique usually combined with the Dent sleeve catheter. See, for example, F. Mearin et al. in “Complete lower esophageal sphincter relaxation observed in some achalasia patients is functionally inadequate”, American Journal of Physiology, vol. 278, no. G376-G383, March 2000; and J. E. Richter in “Diagnostic Test for Gastroesophageal Reflux Disease”, American Journal of Medical Sciences, vol. 326, no. 5, pp. 300-8, November 2003. In this method, the Dent sleeve catheter is constantly perfused with water at the proximal end, and the distal end is open to vent the perfusate. Contraction of sphincter squeezes the Dent sleeve catheter to cause increased resistance to the flow of water. Positioning the Dent sleeve in the LES and continuously measuring the pressure allows monitoring the sphincter function in LES.

These conventional techniques are able to detect the dysfunction of sphincters. However, their data are indirect and exhibit numerous drawbacks related to accuracy and sensitivity. In addition, these methods are unable to provide quantitative analysis about the levels of the opening or the closing of the sphincters.

Therefore, there is a need for a device and a method for directly and/or quantitatively measuring the level of opening and closing of the sphincters. There is also a need for a device and a method for monitoring the function of different GI sphincters using minimally invasive techniques to minimize any external influence.

### SUMMARY OF THE INVENTION

Some aspects of the invention provide a device for sensing/monitoring gastrointestinal tract sphincters and methods for using the same to monitor sphincter function. The invention also provides a system-level software and/or hardware for the implementation and the application of methods disclosed herein.

Some devices of the invention are wave-based sensing devices that can sense/monitor the opening and the closing of GI sphincters. Devices of the invention are minimally invasive.

In some embodiments, the device of the invention uses wave signal measurements inside the GI tract of a patient to monitor the opening and the closing of GI sphincters for the purpose of diagnosing gastrointestinal diseases. The device can be implemented as a stationary system, a semi-ambulatory system (effective within a limited perimeter) or a completely ambulatory system.

Yet in other embodiments, the device of the invention can include a computer-readable medium, and software stored on the computer-readable medium adapted to be executed by a processor. Such a system allows automated monitoring and/or interpretation of wave signal measurements.

Other aspects of the invention provide a method for using the device disclosed herein for monitoring the opening and closing of sphincter, e.g., LES. One particular embodiment of the invention provides a semi-ambulatory wave-based system for monitoring the level of opening of a GI sphincter.

### BRIEF DESCRIPTION OF THE DRAWINGS

FIG. 1 is a general flow chart of a system incorporating a device of the invention to monitor the function (e.g., opening and closing dynamics) of a GI sphincter in a patient.

FIG. 2 is a schematic illustration depicting one method of implementing the device of the invention for a stationary system to monitor the function of a GI sphincter, e.g., the LES between the esophagus and the stomach.

FIG. 3 is a schematic illustration of another method of implementing the device of the invention for a semi-ambulatory system to monitor the function of a GI sphincter while allowing the patient to have limited mobility during the monitoring.

FIG. 4 is a schematic illustration depicting yet another method of implementing the device of the invention for a completely ambulatory system to monitor the function of LES.

FIG. 5 is a picture of a GERD simulation model that was used to validate one method of implementing a semi-ambulatory device of the invention.

FIG. 6 is a flow chart of the monitoring algorithm of a limited ambulatory device of the invention.

FIG. 7 is a plot of the level of modeled LES opening vs. time over 15 minutes during which the modeled LES randomly opened and closed.

### DETAILED DESCRIPTION OF THE INVENTION

The invention will be described with regard to the accompanying drawings which assist in illustrating various features of the invention. In this regard, the invention generally relates to devices and methods for sensing and/or monitoring function of gastrointestinal tract sphincters. Such devices and methods can be used to determine whether a particular sphincter is functioning properly or to diagnose a disease or a clinical condition associated with abnormal function of a sphincter. In particular, the invention relates to a signal-based sensing device that can sense and/or monitor the opening and the closing of GI sphincters. It should be noted that any type of signal can be used in devices and methods of the invention. However, for the sake of clarity and brevity, the invention will be described in reference to a wave-based device.

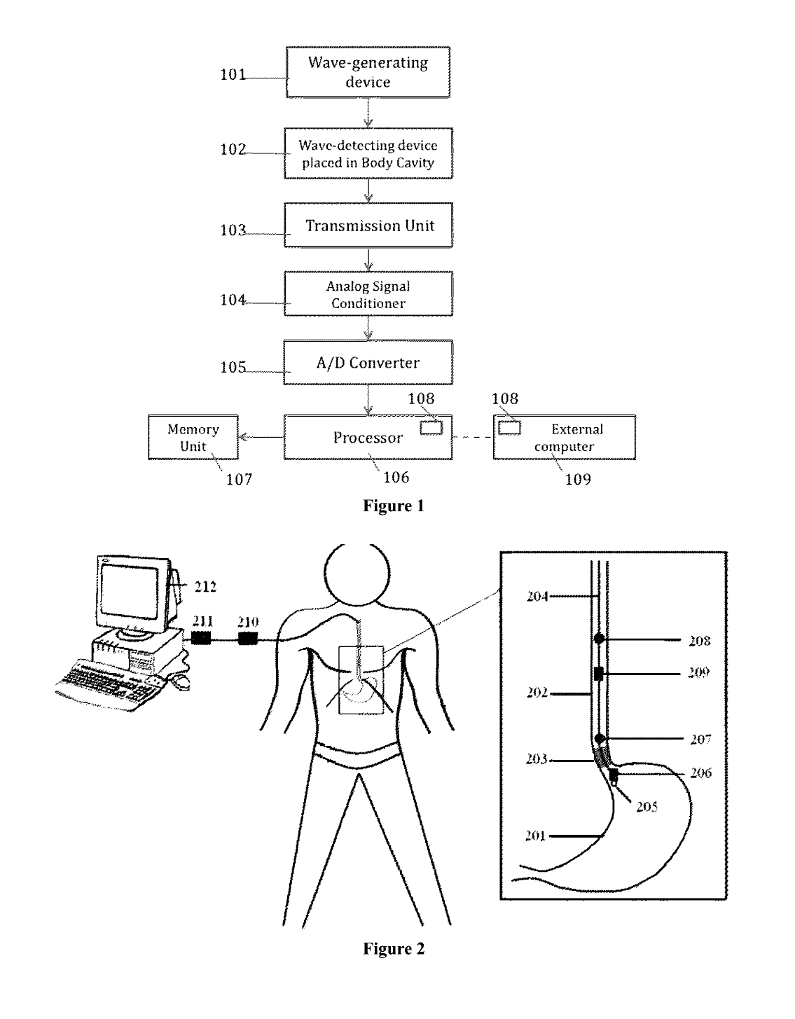

One particular aspect of devices and methods of the invention are illustrated as a flow diagram in FIG. 1. Three particular embodiments of GI tract sphincter monitoring devices are schematically illustrated in FIGS. 2-4. The term “monitoring” includes quantitative and/or qualitative determination of opening and closing of a sphincter whose function is to be monitored. The term can also include the frequency of opening and closing, the duration of opening and/or closing of a sphincter, as well as any other properties that is associated with a given sphincter. It should be noted that all drawings presented herein are provided merely for the purpose of illustrating the practice of the present invention and do not in any way constitute limitations on the scope of the invention.

Devices of the invention provide a novel monitoring modality by recording wave signals at one side of a GI sphincter that are generated by a wave-generating device located at the other side of the GI sphincter over certain time periods. Wave detection can be acquired using a computer or any other devices that are capable of detecting the wave generated by the wave-generating device. Detected wave can be either stored (i.e., logged) for future interpretation/analysis or it can be displace in real-time, or both. Generally, the detected wave is processed to produce a useful information. Such a process generally utilizes a device comprising a central processing unit, such as a computer.

FIG. 1 shows a block diagram or a flow chart of one particular embodiment of the invention. A wave-generating device 101 (i.e., signal-generating device) is placed at one side of a GI sphincter within the GI tract and generates a signal with a fixed frequency, or a specific frequency spectrum pattern, which is captured by at least one wave-detecting device 102 (i.e., signal-detecting device) that is placed on the other side of the sphincter to be monitored. Thus, the wave-generator 101 and the wave-detecting device 102 are placed within the interior of the gastrointestinal tract on the opposite side of the sphincter whose function is to be monitored.

Wave-generating device 101 can be configured to generate any necessary wave desired. For example, the wave-generating device 101 can be an acoustic oscillator. In one embodiment, device of the invention comprises two corresponding wave-detecting devices (e.g., microphones). The first wave-detecting device is placed on one side of the GI sphincter, typically to sphincter's immediate vicinity (e.g., within 5 cm or less, typically within 4 cm or less, often within 3 cm or less, and most often within 1 cm or less), to capture the signal from the wave-generating device that is located at the other side of the sphincter, while the second wave-detecting device (e.g., another microphone) is distantly positioned (e.g., more than 5 cm or alternatively, at least 1 cm, typically at least 2 cm, and often at least 3 cm further away from the first wave-detecting device) along the GI tract. The second wave-detecting device is often used to capture noise signal for adaptively removing the background noise from the wave-signal. Such noises include, but may not be limited to, heartbeat, respiration artifacts and other possible environmental noises or reference signals for wave-signal calibration. Then, a transmission unit 103 receives the signals from the above-mentioned wave-detecting devices 102 and transmits the wave signal to the display, a central processing unit, a signal storage device, or other suitable devices or a combination thereof. The transmission unit 103 can simply be electrical wire, or a wireless transmitter-receiver pair. The wave-detecting device 102 can detect the wave signal in analog or digital form. When the wave-detecting device 102 detects the wave signal in an analog form, the analog signals from wave-detecting device 102 can be transmitted through the transmission unit 103 to an analog signal conditioner 104, an analog-to-digital (A/D) converter 105, with the digitized signals reaching a processor 106. It should be appreciated that the wave signal can be displayed either in the analog form or a digital form. The actual form does not matter as long as the information is useful in sensing/monitoring the function of sphincter.

The processor 106 can be a part of a computer that can display and further analyze the signals under the control of a custom-designed software package during a test. Alternatively, the processor 106 can be part of a data logger (for example, a microcontroller) equipped with the necessary storage memory 107 in which the digitized signals are stored for future processing and evaluation. A standard distributed interface 108 can be embedded with the processor 106. The portable data logger 108 optionally can be connected to an external computer 109, e.g., through the standard distributed interface 108 during an ambulatory testing. The signal processing can take place either in real-time or off-line.

TABLE 1 below lists device of the invention utilizing several different wave-based technologies. The generated and detected waves can include, but are not limited to, acoustic wave signal, ultrasonic wave signal, radio-frequency wave signal, optical wave signal etc., or a combination of two of more of such waves.

TABLE 1
Devices of the invention utilizing four different
exemplary wave processing technologies.

| Type | Wave-generating device | Wave-detecting device |
|---|---|---|
| Acoustic wave | Oscillator | Microphone |
| | (e.g. CX-2V, STATEK, | (e.g. B6W4, Countryman, |
| | Orange, CA, USA) | Menlo Park, CA, USA) |
| Ultrasonic wave | Ultrasound transducer | Ultrasound transducer |
| | (e.g. DC6, Mindray, | (e.g. DC6, Mindray, |
| | Shenzhen, China) | Shenzhen, China) |
| Radio-Frequency | RF transmitter (e.g. | RF receiver |
| wave | 433 MHz, MX-FS-03 V, | (e.g. 433 MHz, MX-05 V, |
| | Geree Technology, | Geree Technology, |
| | Shenzhen, China ) | Shenzhen, China) |
| Optical wave | LED | Optical Receiver |
| | (e.g. LXZ1, Philips | (e.g. Optical Receiver, |
| | Lumileds, San Jose, | PHOTONIS, Frisco, TX, |
| | CA, USA) | USA) |

In one particular embodiment, which is used herein merely as an illustrative example, the wave-generating device 101 is an acoustic oscillator, which can include a quartz crystal integrated circuit that is routinely utilized to generate a stable sound signal. For example, the CX-2V (e.g. STATEK, Orange, Calif., USA) quartz crystal oscillator can be used for this purpose. The wave-detecting device 102, correspondingly, can be a B6W4 microphone (available from, for example, Countryman, Menlo Park, Calif., USA), which is of small size and provides high signal-to-noise ratio, high sensitivity and good ambient noise-shrouding capability. The wave-generating device 101 and the wave-detecting device 102 can be installed on a catheter and inserted into the gastrointestinal tract of a subject. Alternatively, the wave-generating device 101 and the wave-detecting device 102 can be separately inserted. The transmission unit 103, which transmits the detected wave, can be one or more electrical wires in stationary and completely ambulatory systems, or a wireless transmitter-receiver pair in semi-ambulatory systems. In general, any device that can transmit the wave-signal detected by the wave-detecting device 102 to an appropriate device can be used.

For the wireless transmitter-receiver pair, two analog transmitters (e.g. UR1, Shure, Niles, Ill., USA) can be employed to collect and transmit the analog signals from the wave-detecting device 102 to two analog receivers (e.g. UR4D, Shure, Niles, Ill., USA). Optionally, the device of the invention can also include an analog signal conditioner 104. The analog signal conditioner 104 can include, one or more of the following: amplifier, filter, transient protection and other circuitry to amplify and condition the analog signals from the transmission unit 103. In particular, the signal conditioner can also include a low-pass filter, a high-pass filter, a band-pass filter, a band-reject filter, or combination thereof to remove undesirable noises or background waves.

Devices of the invention can optionally include an analog to digital signal converter (“A/D converter”) 105. The A/D converter 105 receives the conditioned analog signals and digitizes them. An exemplary commercially available A/D converter, which should not be considered limiting, is the NI-9234 (available from National Instruments, Austin, Tex., USA) for high-speed data acquisition in a bandwidth that embeds the bandwidth of the wave signal generated by the wave-generating device 101. It should be appreciated that the wave-detecting device 102 can be an integrated unit that includes one or more of the transmission unit 103, analog signal converter 104, and A/D converter 105. Moreover, device of the invention can also omit one or more of the transmission unit 103, analog signal conditioner 104 and A/D converter 105.

The digitized signals are then processed by the processor 106. In stationary systems, the processor 106 can be a computer that is installed with specific task handling software or program. The software can include one or more software routines that are implemented using any of a variety of programming techniques and languages without departing from the scope and the spirit of the invention. One commonly available software that is suitable is the NI Sound and Vibration Toolkit (National Instruments, Austin, Tex., USA), which can display graphical information related to the wave signals detected by the wave-detecting device 102. In ambulatory systems, the processor 106 can be a commercially available microcontroller (e.g. PIC18F8X20, Microchip Technology, Chandler, Ariz., USA) which allows the controller to access external memory devices (such as Flash, EPROM, SRAM, etc.) as program or data memory. The collected signals stored in the memory unit 107 during the test can later be transferred to the external computer 109 for offline display, data processing and analysis. Thus, the information extracted from the computer can facilitate medical personnel in monitoring the actual opening and closing the interrogated GI sphincter at their leisure, and relate these observations to a particular GI disease, for example, but not limited to, GERD, pyloric stenosis, ileocecal abnormalities, etc.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Embodiment 1

FIG. 2 is a diagram depicting one embodiment as a stationary system incorporating the device of the invention that includes a wave-generating device and a wave-detecting device. This embodiment illustrates monitoring of the opening and closing of lower esophageal sphincter (LES) 203, one of the GI sphincters that is located at the lower end of the esophagus 202, before the latter connects to the stomach 201. This embodiment adopts a catheter-based system, in which an optional pH sensor 205 is mounted at the distal end of a catheter 204. The signal detected by the pH sensor 205 is transmitted through an electrical wire inside the catheter 204 and out of the body of the patient.

The reading of the pH sensor 205 is utilized to indicate whether the tip of the probe is properly anchored in the stomach when the catheter is being transorally or transnasally inserted in the esophagus 202. An acoustic oscillator 206 is mounted a few millimeters proximally to the pH sensor 205 on the catheter 204. Once activated, the oscillator 206 can generate sound signals having desired characteristics, for example, a fixed frequency or a specific bandwidth. A first microphone 207 is mounted a few centimeters proximally to the oscillator 206 on the catheter 204 to form a distal wave-generating and detecting pair. The positions of the oscillator 206 and the microphone 207 in this distal pair are interchangeable. A second oscillator 209 and a second microphone 208 are mounted a few centimeters proximal to the first wave-generating and detecting pair to serve as a reference pair. The positions of the oscillator 209 and the microphone 208 in this reference pair are also interchangeable. The position of the catheter 204 relative to the LES 203 can be adjusted by sliding the catheter 204 along the esophagus 202. Typically, the oscillator 206 is placed or located right below the LES 203 and the first microphone 207 is placed or located right above the LES 203. The first microphone 207 can capture sound signals having one or multiple underlying dynamic characteristics, for example, the power of sound signals at the fixed frequency or bandwidth generated by the oscillator 206, when the LES 203 is opening or closing over a long period of time. Meanwhile, the second microphone 208 captures noises or background waves from other inner organs and environment, e.g., the heart beats and/or respiration artifacts for adaptive filtering and reference signals for sound signal calibration, when the oscillator 209 is activated. The analog signals from the first microphone 207, the second microphone 208 and the pH sensor 205 are individually transmitted through separate electrical wires to a multichannel analog signal conditioner 210 which amplifies and conditions the received analog signals. The analog signals are then digitized by a multichannel analog-to-digital (A/D) converter 211. The digitized signals are continuously stored and displayed on a computer 212 in real time.

The mobility of the patients is restricted while utilizing stationary diagnostic systems. This may cause discomfort to some patients and potentially reduce the level of patients' acceptance of these systems. Also with these stationary tests, the monitoring time is relatively short and is not performed in natural conditions during the normal daily routine of the patient. Although this embodiment was illustrated utilizing acoustic wave sensing, it should be appreciated any type of wave, e.g., those listed in Table 1, signal can be used.

### Embodiment 2

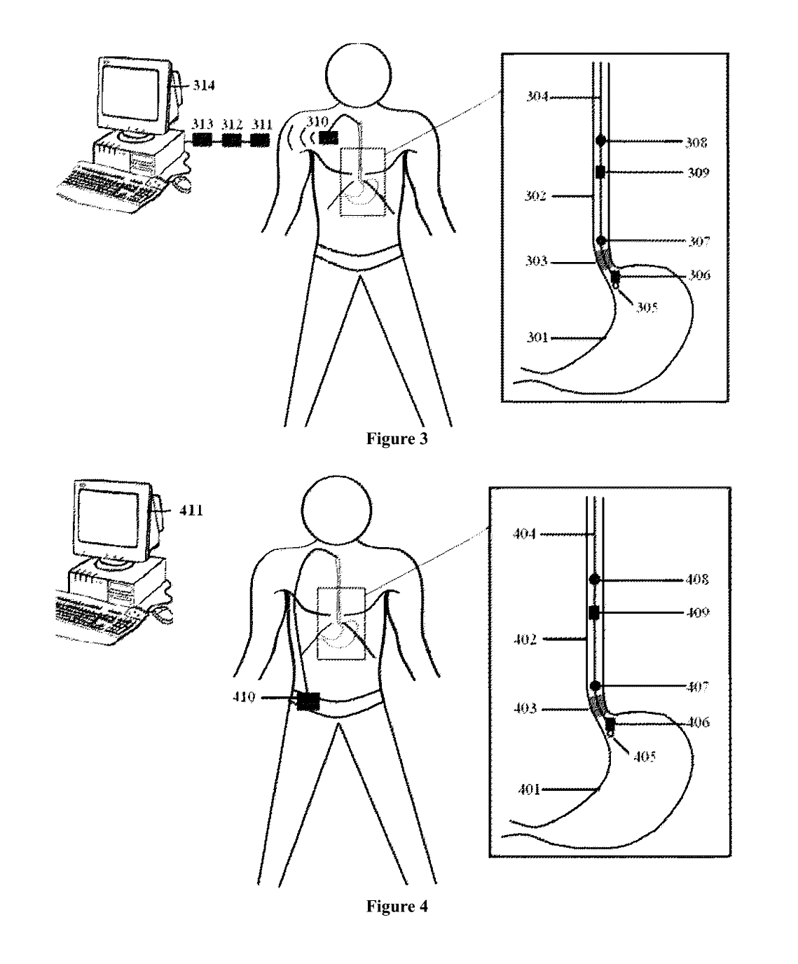

FIG. 3 illustrates another embodiment of the invention. In particular, FIG. 3 shows a semi-ambulatory system incorporating the wave-sensing device disclosed herein. This particular embodiment also illustrates a catheter-based system, in which a pH sensor 305 is mounted at the distal end of a catheter 304. The signal detected by the pH sensor 305 is transmitted through an electrical wire inside the catheter 304 out of the body of the patient. The reading of the pH sensor 305 is utilized to indicate whether the tip of the probe is properly placed, located or anchored in the stomach when the catheter is transorally or transnasally inserted in the esophagus 302.

An oscillator (i.e., a wave signal generator) 306 is mounted a few millimeters proximally to the pH sensor 305 on the catheter 304. Once activated, the oscillator 306 can generate sound signals or other suitable wave signals having desired characteristics, for example, but not limited to, a fixed frequency or a specific bandwidth. A first wave-detector (e.g., a microphone, if the oscillator generates sound wave) 307 is mounted a few centimeters proximally to the oscillator 306 on the catheter 304 to form a distal wave-generating and detecting pair. The positions of the oscillator 306 and the microphone 307 in this distal pair are interchangeable. Optionally, a second oscillator 309 and a second microphone 308 are mounted a few centimeters proximal to the first wave-generating and detecting pair to serve as a reference pair. The positions of the oscillator 309 and the microphone 308 in this reference pair are also interchangeable. The position of the catheter 304 related to the LES 303 can be adjusted by sliding the catheter 304 along the esophagus 302. At its typical application position, the oscillator 306 is located right below the LES 303 and the first microphone 307 is located right above the LES 303. The first microphone 307 can capture sound signals having one or multiple underlying dynamic characteristics, for example, the power of sound signals at the fixed frequency or bandwidth generated by the oscillator 306, when the LES 303 is opening or closing over a long period of time. Meanwhile, the second microphone 308 captures noises from other inner organs and environment, e.g., the heart beats and respiration artifacts for adaptive filtering and reference signals for sound signal calibration, when the oscillator 309 is activated.

The analog signals from the first microphone 307, the second microphone 308 and the pH sensor 305 are individually transmitted through a wireless transmission unit, which comprises an analog transmitter 310 and an analog receiver 311, to an analog signal conditioner 312 which amplifies and conditions the received analog signals. The wireless transmission between analog transmitter 310 and analog receiver 311 allows patient to walk around within a certain distance during the monitoring, which defines the system as semi-ambulatory. The analog signals are then optionally digitized by an analog-to-digital (A/D) converter 313. The digitized signals can be continuously stored and/or displayed on a computer 314.

### Embodiment 3

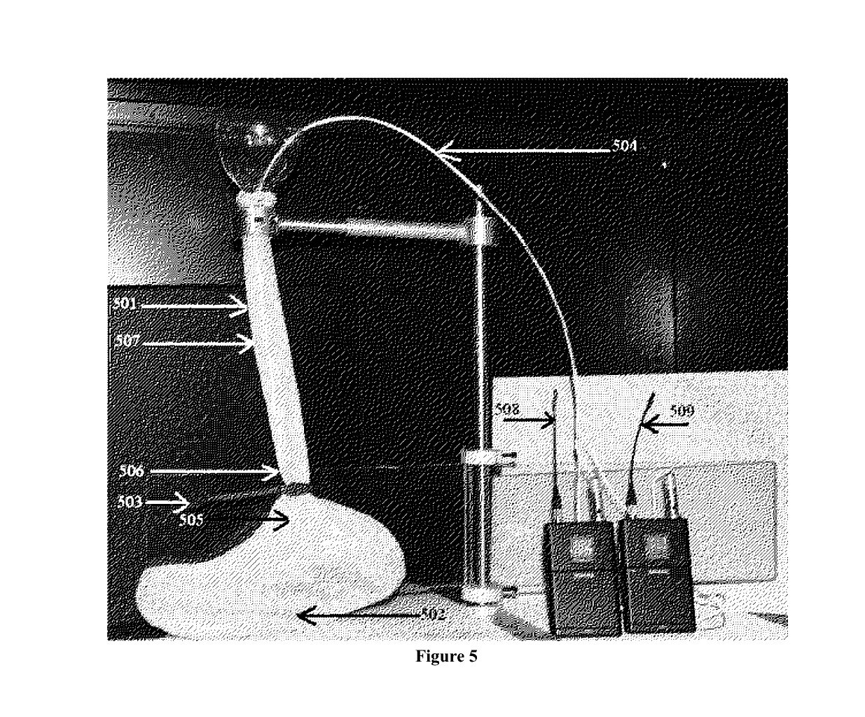

Still in another embodiment, an ambulatory system of the invention is presented to provide a comfortable solution to patients. FIG. 4 is an illustration depicting the third embodiment of the invention. As shown in FIG. 4, the device is positioned to monitor the opening and closing of lower esophageal sphincter (LES) 403, one of the GI sphincters that is located at the lower end of the esophagus 402 and meets the stomach 401.

As shown in FIG. 4, this embodiment also utilizes a catheter-based system. As with other embodiments, the device illustrated in FIG. 4 can also optionally include a pH sensor 405 that is mounted at the distal end of a catheter 404. The signal detected by the pH sensor 405 is transmitted through an electrical wire inside the catheter 404 out of the body of the patient. One of the reasons for including the pH sensor 405 is to indicate whether the probe is properly placed, located and/or anchored in the stomach when the catheter is transorally or transnasally inserted via the esophagus 402. An oscillator 406 is mounted a few millimeters proximal to the pH sensor 405 on the catheter 404. Once activated, the oscillator 406 generates sound signals having desired characteristics, for example, a fixed frequency or a specific bandwidth. A first microphone 407 is mounted a few centimeters proximally to the oscillator 406 on the catheter 404 to form a distal wave-generating and detecting pair. The positions of the oscillator 406 and microphone 407 in this distal pair are interchangeable. A second oscillator 409 and a second microphone 408 are mounted a few centimeters proximal to the first wave-generating and wave-detecting pair to serve as a reference. The positions of the oscillator 409 and the microphone 408 in this reference pair are also interchangeable. The position of the catheter 404 related to the LES 403 can be adjusted by sliding the catheter 404 along the esophagus 402. At its typical operational position, the oscillator 406 is located right below the LES 403 and the first microphone 407 is located right above the LES 403. The first microphone 407 can capture sound signals having one or multiple underlying dynamic characteristics, for example, the power of sound signals at the fixed frequency or bandwidth generated by the oscillator 406, when the LES 403 is opening or closing over a long period of time. Meanwhile, the second microphone 408 captures noises from other inner organs and environment, e.g., the heart beats and respiration artifacts for adaptive filtering and reference signals for sound signal calibration, when the oscillator 409 is activated. The analog signals from the first microphone 407, the second microphone 408 and the pH sensor 405 can be individually transmitted to the portable data logger 410 through multiple electrical wires. The analog signals are subsequently conditioned, digitized and stored in the data logger 410. When the patient completes the ambulatory test, the data stored in the data logger 410 is then transferred to a computer 411 for further offline processing and analysis.

### Analysis Method

The method associated with the analysis of the data obtained from the device of the invention includes the processing of the wave signals captured by the wave-detecting device of the distal pair which is distributed in the immediate vicinity of the GI sphincter (hereafter called primary signal) as well as the wave signals captured by the wave-detecting device in the reference pair (hereafter called reference signal), positioned away from the said sphincter. In one particular method, the primary signal and the reference signal are sent to an adaptive filter in order to minimize or reduce the noise and to retain only the desired signal or to increase the desired signal that corresponds to a particular feature of the GI sphincter that is being monitored. Then, at least one desired dynamic characteristic of the signal, for example, the power of the signal in the adaptively filtered primary signal, can be utilized to represent the opening and closing dynamics of the GI sphincter of interest. The adaptive filtering algorithm based on a primary signal containing signal s1 contaminated with noise n1, and a reference signal containing noise n2, correlated with the noise n1 in the primary signal, has been described in the literature (see e.g. Haykin S and B. Widrow (Eds), Least-Mean-Squares Adaptive Filters, John Wiley, 2003; ISBN 0-471-21570-8).

### In-Vitro Simulation

To test the relationship between the percentages of the LES opening and the strength of the sound signals, an experimental model was built to simulate the motility of the distal esophagus as well as the gastroesophageal reflux dynamics. FIG. 5 is an illustration depicting the components of the simulation model. The model included a silicone stomach model 502 (Simulab Corporation, Seattle, Wash., USA) having a model esophagus 501. The stomach model 502 was filled with physiological saline to simulate gastric content. For testing, the catheter 504 was inserted into the experimental model to monitor shape changes. A plastic seal 503 with labeling is employed to control the percentage of the LES opening. The purpose of the prototype which implemented a model of one of the embodiments, embodiment 2 (semi-ambulatory system), was to evaluate the performance of the acoustic catheter 504 and to determine its suitability for in vitro testing in a real esophagus. The positions of the oscillator 505, microphone 506 and microphone 507 are marked in the figure. Modules 508 and 509 transmit the output signal from the microphones to receivers (not shown in the figure). Using this model, measurements from the acoustic system were correlated to the percentage of simulated LES opening. Although the material of the plastic seal 503 is different from the actual composition of the esophagus, the opening and the closing of the LES were effectively simulated in this model.

In order to study the relationship between the degree of the LES opening and the strength of the sound at the microphone, eleven groups of data were collected in a quiet environment and another eleven groups of data were collected in a noisy environment. Ten different percentages were introduced. “0”, meaning LES is totally open, “10”, meaning LES is 10% closed, “20”, meaning LES is 20% closed, and so on. “100” meant LES was totally closed. For tests using this in vitro simulation model, the sound strength when the LES was totally open was known. However, for in vivo tests, the sound strength when the esophagus is totally open needs to be determined experimentally.

In the first trial, data was collected in a quiet environment. To test the robustness of this method, in the second trial, same test incorporating the same procedures was performed with an addition of random noise during the recording. The noise came from purposely stirring a bottle of water near the crystal in order to mimic the sounds from gastric juice movements in the stomach. Before the experiment, the oscillator circuit was turned on and the distance between the oscillator and the lower microphone was fixed at 5 cm. For each recording, the sound signal was recorded for 30 seconds. Then, the plastic seal was tightened to the next level to get the LES more closed and the recording was repeated.

Pearson's correlation was applied to study the relationship between the signal strength and the level of LES closing:

$$r = \frac{{\sum }_{i = 1}^{n} ( {X}_{i} - \overline{X} ) ( {Y}_{i} - \overline{Y} )}{\sqrt{{\sum }_{i = 1}^{n} {( {X}_{i} - \overline{X} )}^{2}} \sqrt{{\sum }_{i = 1}^{n} {( {Y}_{i} - \overline{Y} )}^{2}}} \qquad ( 1 )$$

where r is the Pearson correlation coefficient, X is one of the monitored variables, and Y is the second. r always lies between −1 and +1. Positive values of r indicate a tendency of X and Y to increase together. When r is negative, large values of X are associated with small values of Y.

Table 2 shows the Pearson correlation coefficients between the level of LES opening and the signal strength at a fixed frequency, which is one of several possible desired wave dynamic characteristics captured by the implemented wave-based device employing an acoustic wave sensing approach in both quiet and noisy environments. The Pearson Correlation Coefficient in both data sets were higher than the lower limit value for the level of significance p<0.05, which indicates that the change of the strength of the sound signal at a specific frequency is significantly correlated with the level of LES opening or closing.

TABLE 2
Pearson correlation coefficients between the level of LES opening
and the sound strength in quiet and noisy environments. Statistical
significance levels at p = 0.05 are also included.

| | In quiet | In noisy | P = 0.05 |
|---|---|---|---|
| Degree of Freedom = 9 | environment | environment | level |
| Pearson correlation | 0.886 | 0.792 | 0.602 |
| coefficient in data1 | | | |
| Pearson correlation | 0.915 | 0.834 | 0.602 |
| coefficient in data2 | | | |

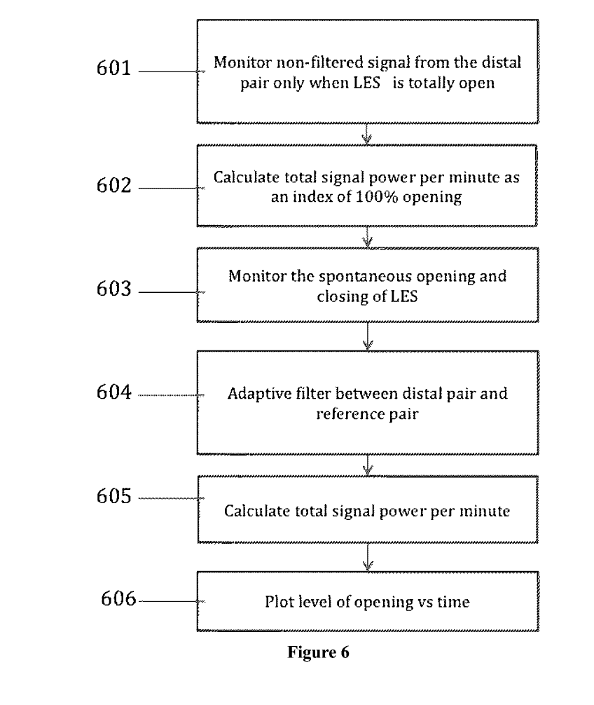

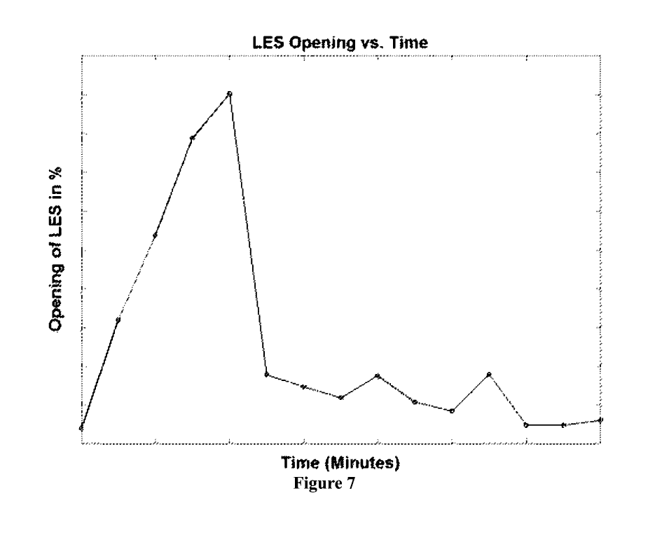

FIG. 6 shows the algorithm for monitoring the opening and the closing of the LES. In Block 601, a series of non-filtered signals from the distal pair when LES was totally open was monitored. The total signal power per minute was calculated and correlated to an index of 100% opening of the LES in Block 602. In Block 603, monitoring the spontaneous opening and closing of LES was performed and signals from both distal and reference pair were measured. In Block 604, an adaptive filter was applied to remove the noises effects from other inner organs and environment. The total signal power per minute was calculated after adaptive filtering in Block 605 and correlated with the corresponding level of LES opening and closing. Lastly, a figure showing the level of opening of LES vs real-time can be obtained. For example, FIG. 7 shows an example recording over 15 minutes during which the LES randomly opened and closed. The peak value indicates the LES is totally open, the trough shows the LES is totally closed. The level of the LES opening can be calculated by scaling the signal power to 100% between the peak and the trough.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for monitoring a sphincter function in the gastrointestinal tract of a subject, said method comprising:
generating a signal within a gastrointestinal tract interior of a subject on one side of a sphincter whose function is to be monitored;
detecting the generated signal within the gastrointestinal tract interior of said subject on the side opposite of the sphincter whose function is to be monitored; and
analyzing the detected signal to determine the functionality of the sphincter.

2. The method of claim 1, wherein the signal comprises acoustic wave, ultrasonic wave, electromagnetic wave, or a combination thereof.

3. The method of claim 2, wherein the electromagnetic wave is a radio wave, infrared wave, visible wave, ultra violet wave, or a combination thereof.

4. The method of claim 1 further comprising the step of reducing the background noise from the detected signal prior to analyzing the detected signal.

5. A method for monitoring a sphincter function in a gastrointestinal tract of a subject, said method comprising:
placing a signal-generating device and a signal-detecting device within the interior of a gastrointestinal tract of a subject such that said signal-generating device is located on one side and said signal-detecting device is located on the other side of a sphincter whose function is to be monitored;
generating a signal using said signal-generating device;
detecting the generated signal using said signal-detecting device; and
analyzing the detected signal to determine the sphincter's function.

6. The method of claim 5, wherein said analyzing step comprises the step of reducing the background noise from the detected signal to produce a refined signal and analyzing the refined signal to determine the sphincter's function.

7. The method of claim 5, wherein the signal is a wave.

8. The method of claim 7, wherein said wave comprises acoustic wave, ultrasonic wave, electromagnetic wave, or a combination thereof.

9. A system for monitoring a sphincter function in a subject comprising:
(i) a combination probe having a distal location and a proximal location, said combination probe comprising:
(a) a flexible tube; and
(b) a signal generator located at said distal location; and
(c) a signal detector located at said proximal location,
wherein said signal generator and said signal detector are located at a sufficient distance apart from one another such that when in use one of said signal generator or said signal detector is placed on one side of a sphincter whose function is to be monitored and the other is located on the other side of the sphincter whose function is to be monitored such that a signal generated by said signal generator is detected by said signal detector;
(ii) means for transmitting the detected signal to a signal processor, wherein said signal processor produces a processed signal from said detected signal; and
(iii) means for displaying said processed signal on a display.

10. The system of claim 9 further comprising a means for storing said processed signal.

11. The system of claim 9 further comprising a guide element located proximal to said signal generator or said signal detector, whereby said guide element is configured to aid in detecting proper placement of said combination probe.

12. The system of claim 11, wherein said guide element comprises a pH sensor.

13. The system of claim 9, wherein said means for transmitting the signal comprises a wireless transmitting device.

14. The system of claim 9, wherein said signal processor comprises analog signal conditioner.

15. The system of claim 14, wherein said signal processor further comprises an analog-to-digital converter.

16. The system of claim 14, wherein said combination probe further comprising a second signal generator and a second signal detector configured for detecting a noise signal, and wherein said signal processor at least partially reduces the noise signal from said detected signal to generate the processed signal.
